(19) United States
(12) Patent Application Publication
(10) Pub. No.: US 2012/0058504 A1
(43) Pub. Date: Mar. 8, 2012

(54) METHODS AND APPARATUS FOR DIELECTROPHORETIC SHUTTLING AND MEASUREMENT OF SINGLE CELLS OR OTHER PARTICLES IN MICROFLUIDIC CHIPS

(75) Inventors: Paul Chi Hang LI, Coquitlam (CA); Yuchun CHEN, Burnaby (CA); Samar Mohammed HAROUN, Vancouver (CA)

(73) Assignee: SIMON FRASER UNIVERSITY, Burnaby (CA)

(21) Appl. No.: 12/916,489

(22) Filed: Oct. 30, 2010

Related U.S. Application Data
(60) Provisional application No. 61/256,829, filed on Oct. 30, 2009.

(51) Int. Cl. C12Q 1/02; C12M 1/00; C12N 13/00; C12M 1/34

(52) U.S. Cl. 435/29; 435/287.1; 435/283.1; 435/173.1

(57) ABSTRACT

The invention relates to a microfluidic device. The microfluidic device comprises a fluid chamber comprising a particle retention region for retaining at least one particle, such as a cell. The microfluidic device also comprises a plurality of electrodes extending into the particle retention region for applying a dielectrophoretic (DEP) force to controllably move the particle within the particle retention region. The invention also relates to methods of using the microfluidic device to controllably move the particle within the microfluidic device and to monitor, observe, or measure a parameter of the particle. The particle movement may be caused by a DEP force and/or a fluidic force.

### RELATED APPLICATION

This application claims priority from U.S. Provisional Patent Application No. 61/256,829 filed 30 Oct. 2009, which is hereby incorporated by reference.

### TECHNICAL FIELD

The present invention relates to methods and apparatus for manipulation and measurement of single cells or other particles. In particular, the present invention relates to microfluidic and dielectrophoretic methods and apparatus for manipulation and measurement of cells or other particles.

### BACKGROUND

Scientific studies often focus on the biological properties and physical characteristics of single cells. For example, the uptake of chemotherapeutic drugs by cancer cells can be investigated by closely examining the properties of a single cell. Single-cell studies are particularly important if only a very small amount of test sample is available, for example in the case of stem cells. Single-cell measurement may also be useful in profiling cell heterogeneity.

In one particular example, a fluorescent label may be attached to a chemotherapeutic drug and the cell under study may be exposed to the drug. The fluorescence of the cell may then be measured over time to show the extent of drug accumulation. The fluorescence of the cell sample can also be compared to the “background” fluorescence of the solution carrying the cell to provide a base-line reading.

Studies of this sort can be conducted in microfluidic chips adapted for retaining a single cell or a small number of cells. The chip may be deployed on the viewing platform of microscope having an adjustable aperture for directing a beam of excitation light on to the surface of the chip. The aperture defines a “detection window” on the chip at a defined location to enable accurate measurement of cellular and background fluorescence or some other parameter. However, difficulties can arise in trapping a selected cell within a microfluidic device and controllably moving it to the detection window or some other target location on the chip.

In some cases it is important to move a single cell or particle into and out of a microfluidic device detection window in single cell studies so that the background environment can be measured over time. For example, the background fluorescence may vary over time and therefore this parameter needs to be measured repeatedly in order to gather accurate baseline readings.

Two methods have previously been used to move a single cell or particle into or out of a microfluidic device detection window. In a first method the cell may be moved or “shuttled” back and forth by microfluidic control¹ and in a second method the microfluidic chip itself may be physically translated². Both methods suffer from technical drawbacks. In the first method, an operator has to frequently adjust the fluid potential of the microfluidic chip in order to move the single cell. This can be inconvenient and tiresome for the operator and difficult to achieve with reliable accuracy. The second method requires the single cell to adhere to the chip surface, such as a glass slide. Therefore, the second method only works with adherent cells, not suspended cells. In the second method, the chip must be translated back and forth for monitoring the fluorescence of the cell and its surrounding solution (for background correction).

The present application relates to a third method for controllably moving a single cell or other particle. This method is based on dielectrophoretic (DEP) force operating on a cell or particle under a non-uniform electric field.

DEP force has been used in a wide number of other applications for moving particles in a non-uniform electric field. So far, the applications include the separation of nonviable cells from viable cells³ or separation of cells from other cells, or other particles such as beads⁴ or other particles'. Some other groups have reported using the DEP force for cell trapping^{6,7,8}. Recently, the DEP force has been used to characterize molecular interactions between molecules and beads⁹ and antibody-antigen interactions between antibody molecules and cells¹⁰ inside microfluidic devices. However, it is believed that DEP force has not previously been used in a microfluidic environment to controllably move single cells or particles for the purposes of fluorescent measurements and the like. Also, in these earlier applications of DEP force for cell trapping, the cells typically move only in a single direction. The present disclosure describes use of the DEP force to controllably move a cell or other particle in one or more directions, or to maintain the cell at a desired target location.

### BRIEF DESCRIPTION OF DRAWINGS

In drawings intended to illustrate various embodiments, but which are not intended to be construed in a limiting manner:

FIG. 1A is a top plan view of a microfluidic chip according to one embodiment of the invention.

FIG. 1B is an enlarged view of an area denoted by dashed lines in FIG. 1A.

FIG. 2A is a side elevational view of the embodiment of FIG. 1A.

FIG. 2B is a cross-sectional view taken along line 2B-2B of FIG. 1A.

FIG. 2C is a cross-sectional view taken along line 2C-2C of FIG. 1A. The dimensions of the microchannels are enlarged to make them more easily visible.

FIG. 3A is a top plan view of a top layer of the microfluidic chip of FIG. 1A.

FIG. 3B is a top plan view of a bottom layer of the microfluidic chip of FIG. 1A.

FIG. 4A is a perspective view of a cell retention portion of the microfluidic chip of FIG. 1A.

FIG. 4B is an enlarged view of the portion of FIG. 4A.

FIG. 5A illustrates an electrode array disposed in a cell retention structure of a microfluidic chip.

FIG. 5B is a cross-sectional view taken along line 5B-5B of FIG. 1B.

FIG. 5C is a cross-sectional view taken along line 5C-5C of FIG. 1C.

FIG. 6 shows (a) how microchannels and DEP electrodes are arranged on an example microfluidic chip; (b) three example electrodes (A, B and C) and a cell retention structure of the chip of FIG. 6(a), and (c) a photograph showing how a microfluidic chip constructed in accordance with the invention and having electrodes externally connected to voltage supplies.

FIG. 7 is an enlarged cross-sectional view of an example electrode comprising two metal layers.

FIG. 8A illustrates one configuration of an electrode array and a cell retention structure.

FIG. 8B illustrates another configuration of an electrode array and a cell retention structure.

FIG. 9 illustrates a further configuration of an electrode array and a cell retention structure.

FIG. 10 illustrates a further configuration of an electrode array and a cell retention structure.

FIG. 11A illustrates a further configuration of an electrode array and a cell retention structure.

FIG. 11B illustrates a further configuration of an electrode array and a cell retention structure.

FIG. 12A illustrates a further configuration of an electrode array and a cell retention structure.

FIG. 12B illustrates a further configuration of an electrode array and a cell retention structure.

FIG. 13 comprises two photographs of two different electrode arrays terminating in cell retention regions of microfluidic chips (indicated as Chip I and II).

FIG. 14 comprises two photographs of two different electrode arrays terminating in cell retention regions of microfluidic chips (indicated as Chip III and IV).

FIG. 15A shows cell movement by DEP force in Chip I of FIG. 13 when voltage is switched between electrodes A-B and B-C.

FIG. 15B shows cell movement by DEP force in Chip II of FIG. 13 when the voltage is switched between electrodes A-B and B-C.

FIG. 16 is an enlarged view showing the dimension of two electrodes of Chip II of FIG. 13.

FIG. 17 shows (a) the movement of a 6-μm fluorescein-labeled polystyrene bead by DEP force in Chip II of FIG. 13 when the voltage is switched between A-B and B-C; (b) the fluorescent measurement of the bead when it was shuttled by DEP force into the detection window (the peak provides the total fluorescence) and out of the detection window (the base-line provides the background).

FIG. 18A shows cell movement by DEP force in Chip II of FIG. 13 when HBSS medium is used. The voltage (9 MHz and 1.6V) is switched between A-B and B-C. A wild-type leukemia cell (CEM) is used.

FIG. 18B shows the cell movement by DEP force in Chip III of FIG. 14 when α-MEM medium is used. The voltage (30 MHz and 14V) is switched between A-B and B-C. A drug-resistant leukemia cell (CEM VLB) is used.

FIG. 19A shows cell movement by DEP force in Chip IV of FIG. 14 when α-MEM medium is used. The voltage (30 MHz and 14V) is switched between A-B and B-C. A drug-resistant leukemia cell (CEM VLB) is used.

FIG. 19B schematically shows the path of cell movement by DEP force in Chip IV according to FIG. 19A.

FIG. 20 shows (a) the movement of a cell (CEM VLB) by DEP force in Chip IV when the voltage is switched between A-B and B-C. The cell is placed in the HBSS medium. Voltage at 30 MHz and 14V is applied. (b) The fluorescent measurement of the cell when it was shuttled by DEP force into the detection window (the peak provides the total fluorescence) and out of the measurement window (the baseline provides the background).

FIG. 21 shows (a) the movement of a cell (CEM VLB in α-MEM medium) by translating the whole chip (with no electrodes). (b) The fluorescent measurement of the adhered cell when the chip was translated into the measurement window (the peak provides the total fluorescence) and out of the measurement window (the baseline provides the background).

FIG. 22 shows the effect of cyclosporine A (CsA, a well-known MDR inhibitor) on DNR accumulation in a MDR leukemia cancer cell using a DEP-based microfluidic chip.

FIG. 23 shows cell shuttling and manipulation using two electrodes, and in combination with fluid control.

FIG. 23B shows cell movement by DEP force in Chip IV of FIG. 14 when α-MEM medium is used. The voltage (30 MHz and 14V) applied to electrodes AB is turned on and off. The electrode C is not used. A drug resistant leukemia cell (CEM-VLB) is used.

FIG. 24A shows a photograph (top plan view) of a DEP-based microfluidic chip comprising a plurality of cell retentions structures (23 cell retentions structures arranged in an array having five rows).

FIG. 24B is a schematic drawing of the FIG. 24A embodiment.

FIG. 24C shows an enlarged view of a fluid chamber of the microfluidic chip in FIG. 24B.

FIG. 25 shows enlarged views of the cell retention structures and DEP electrodes of FIG. 24B.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

This application relates to a methods and apparatus for controllably moving at least one particle using dielectrophoretic (DEP) force. In this disclosure, the term “particle” includes a cell as well as a non-cellular discrete unit, such as an inorganic or polymeric bead. The invention is particularly adapted for controllably moving a single cell within a microfluidic device, such as a microfluidic chip 10, to enable measurement and analysis of the biological properties or physical characteristics of the cell.

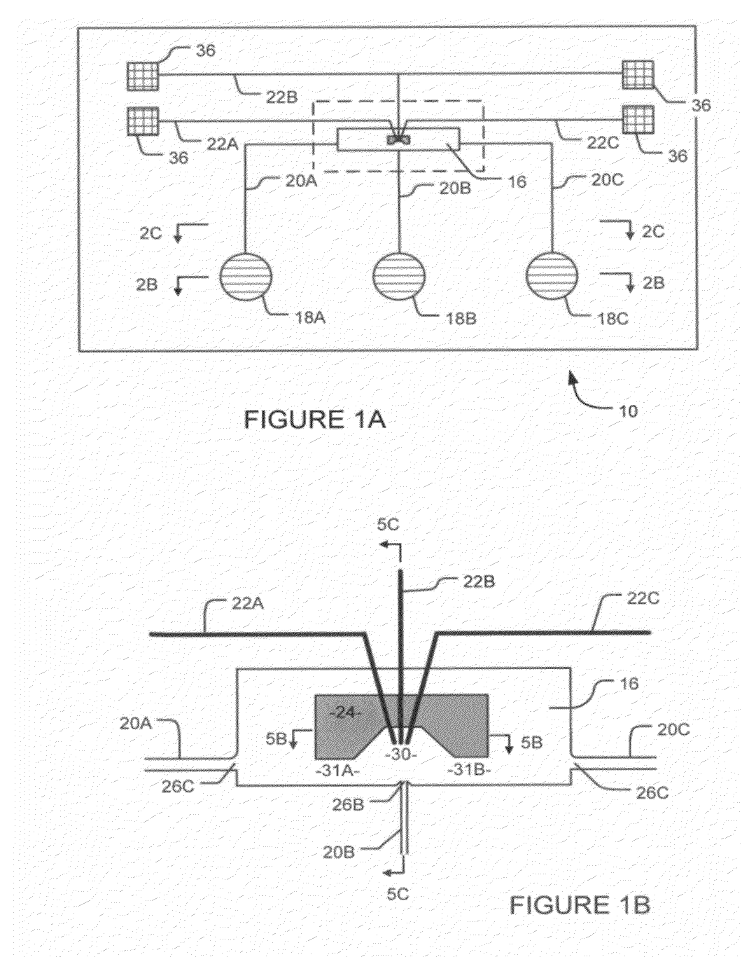

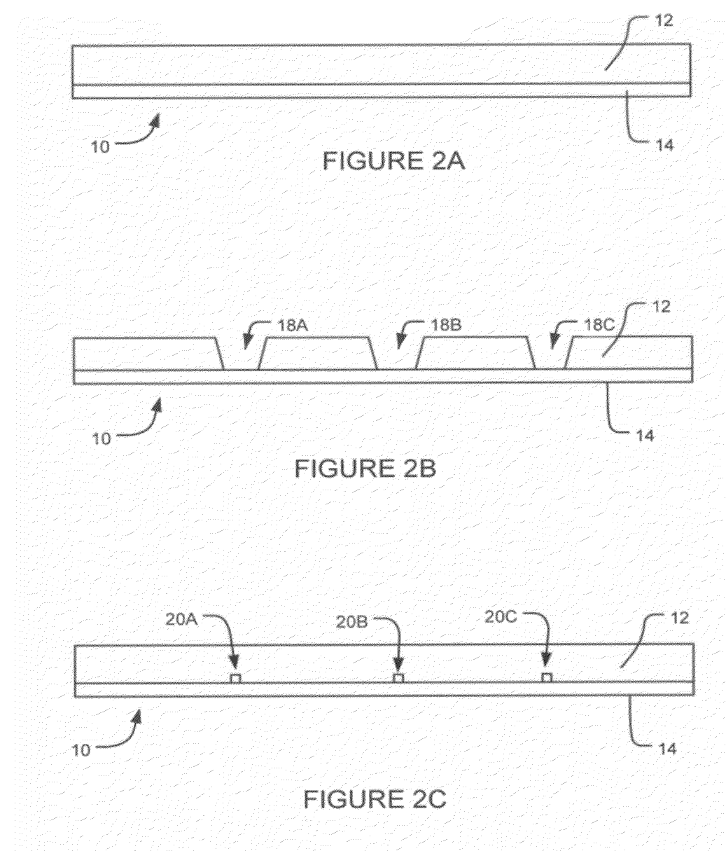

One exemplary embodiment of a chip 10 constructed in accordance with the invention is shown in FIGS. 1-6. In this embodiment chip 10 is formed from two separate layers, namely a top layer 12 and a bottom layer 14 which are bonded together (FIG. 2A). Each of top and bottom layers 12, 14 has an upper surface and a lower surface. In one embodiment top and bottom layers 12, 14 may be made of transparent glass. Since top and bottom layers 12, 14 are transparent, it is possible to see the internal structures of chip 10, as illustrated for example in the top plan view of FIG. 1A, even though some structures may be located on bottom layer 14 or on the lower surface of top layer 12.

In this example, the thickness of the top layer 12 is about 1 mm and the thickness of the bottom layer 14 is about 0.7 mm. In one exemplary embodiment, the dimensions of chip 10 are approximately 15 mm×30 mm. The dimensions may vary without departing from the invention.

As will be appreciated by a person skilled in the art, chip 10 may be constructed of other combinations of layers or materials in other embodiments of the invention. For example, chip 10 may be constructed as a unitary piece. Layers 12, 14 may be optionally constructed of silicon, quartz, plastics, or PDMS.

As shown in FIG. 1A, chip 10 comprises a fluid chamber 16, a plurality of solution reservoirs 18A-C (collectively 18), and a plurality of microchannels 20A-C (collectively 20) for connecting each reservoir 18 to fluid chamber 16. As described further below, a plurality of electrodes 22A-C (collectively 22) are also provided for inducing the application of a DEP force to a cell or other particle introduced into fluid chamber 16.

Fluid chamber 16 may be defined by a relieved portion formed on a lower portion of top layer 12. It is also posible for fluid chamber 16 to be formed on an upper portion of bottom layer 14. In the illustrated embodiment, fluid chamber 16 is located in the center of chip 10, but this is not required. As indicated above, fluid chamber 16 is in fluid communication with solution reservoirs 18 via microchannels 20. Microchannels 20 may be relieved channels formed on the lower portion of top layer 12, or the upper portion of bottom layer 14, or both. Solution reservoirs 18 may also comprise relieved portions formed in top layer 12, and optionally bottom layer 14, or both, and may extend entirely through top layer 12 to form apertures therein, as best shown in FIG. 2B. In the illustrated embodiment reservoirs 18 are round, but other shapes and configurations are possible. FIG. 2B shows a cross section of the three solution reservoirs 18A-C, and FIG. 2C shows a cross section of the three microchannels 20A-C. In FIG. 2C, microchannels 20A-C have been enlarged to make them clearly visible.

Microchannels 20A-C each consist of an elongated cavity formed between top layer 12 and bottom layer 14 which is not directly exposed to the ambient environment. Similarly fluid chamber 16 is a larger enclosed cavity formed between top layer 12 and bottom layer 14. Solution reservoirs 18A-C are also adapted for holding fluid, but they are exposed to the ambient environment. As described further below, solutions or reagents can be added to one of solution reservoirs 18A-C which may then be delivered to fluid chamber 16 via one of microchannels 20A-C. For example, cells may be added via solution reservoirs 18A or 18C and drugs or other reagents may be added via solution reservoir 18B.

In one embodiment some component parts of chip 10 may be made by a process called photolithography which is well-known to those skilled in the art. In particular, fluid chamber 16, microchannels 20 and solution reservoirs 18 may be made by etching top layer 12 or bottom layer 14 or both. In one exemplary embodiment the depth of fluid chamber 16 and microchannels 20 is in the range of approximately 20 to 40 p.m.

FIG. 1B is an enlarged view of an area of chip 10 denoted by dashed lines in FIG. 1A. In this embodiment a cell retention structure 24 is located in fluid chamber 16. Cell retention structure 24 is raised relative to the relieved basin of fluid chamber 16. For example, cell retention structure 24 may be integral with top layer 12 or bottom layer 14 and may comprise an un-etched portion of top layer 12 or bottom layer 14 surrounded by an etched portion thereof (i.e., the etched portion forms fluid chamber 16).

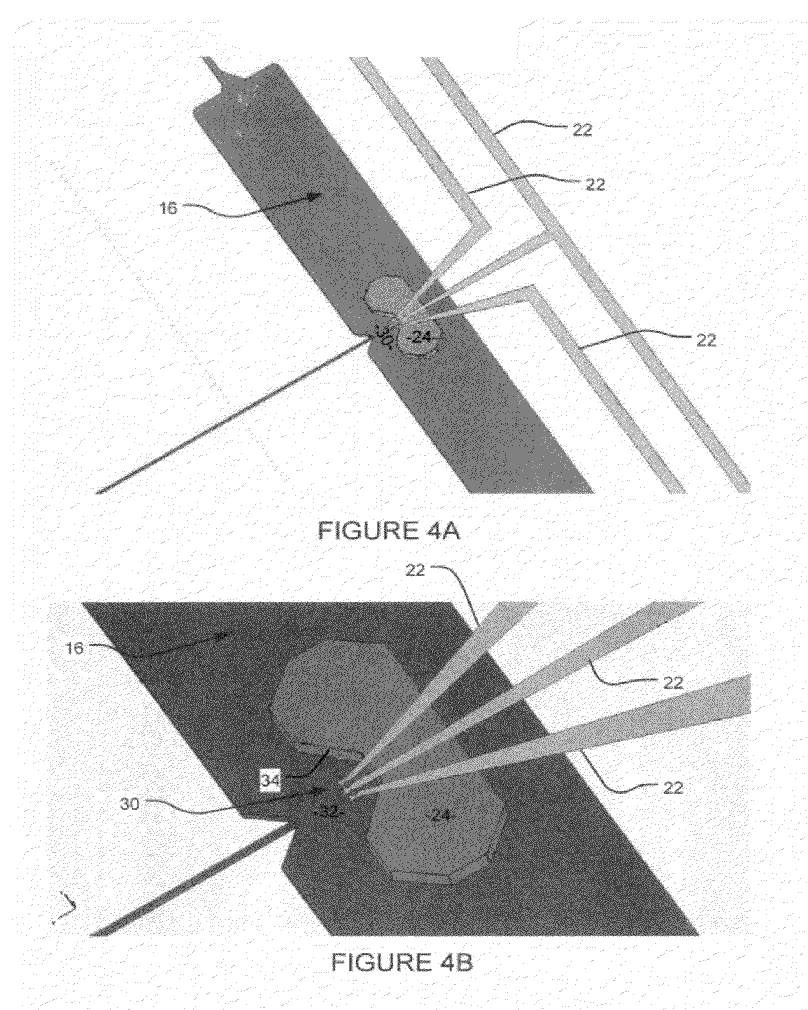

FIG. 1B shows that chamber ports 26A-C (collectively 26) connect microchannels 20A-C to fluid chamber 16. Cell retention structure 24 has a generally recessed V-shaped or U-shaped cell retention portion, which is spaced apart from the middle chamber port 26B and which defines a cell retention region 30 within fluid chamber 16. In FIG. 1B, cell retention region 30 is generally shaped like an isosceles trapezoid, although this is not mandatory. Cell retention region 30 is in fluid communication with the rest of fluid chamber 16 via fluid ports 31A, 31B (collectively 31). Cell retention region 30 has a bottom surface 32 defined by an upper surface of bottom layer 14 and is partially enclosed by side walls 34 of cell retention structure 24 (FIG. 4B). Side walls 34 may be vertical or sloped. Embodiments of microfluidic cell retention structures are described in detail in International Patent Application Publication WO 2006/007701 which is hereby incorporated by reference in its entirety.

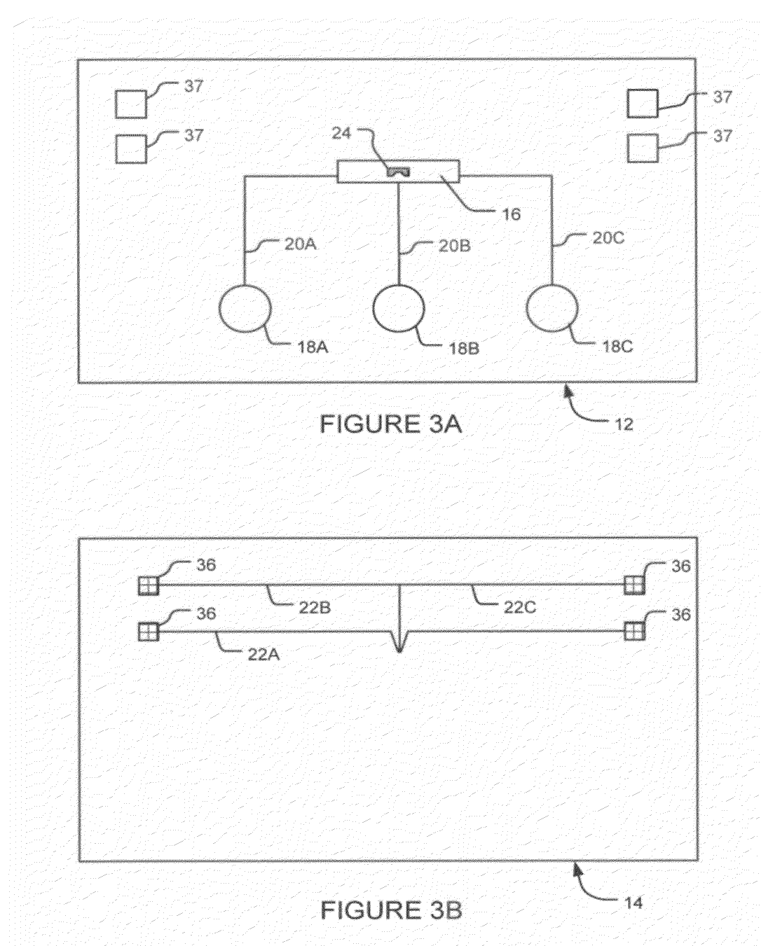

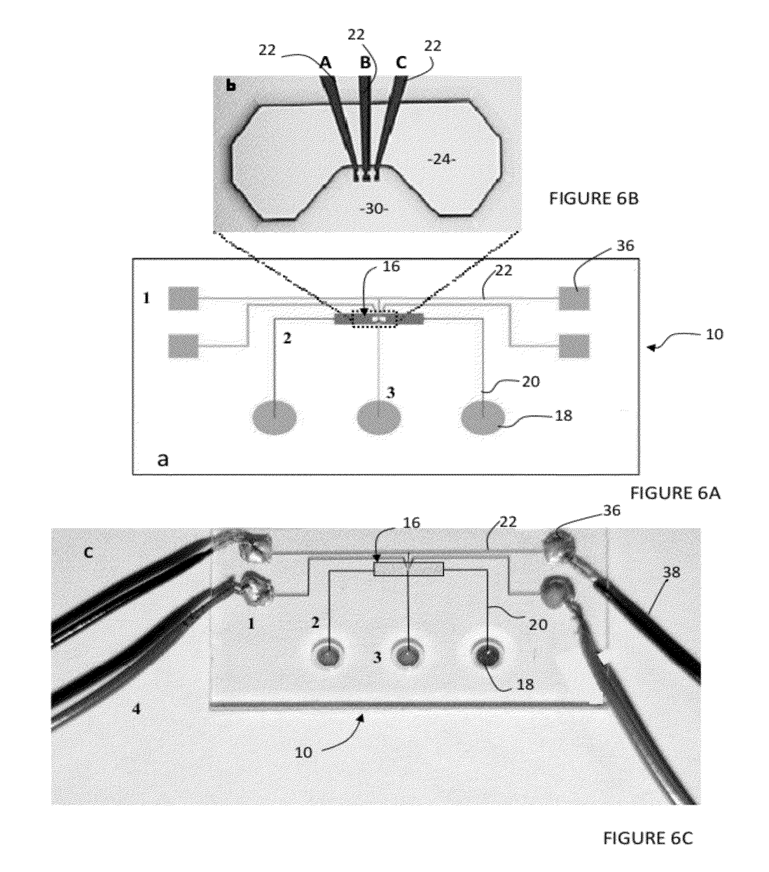

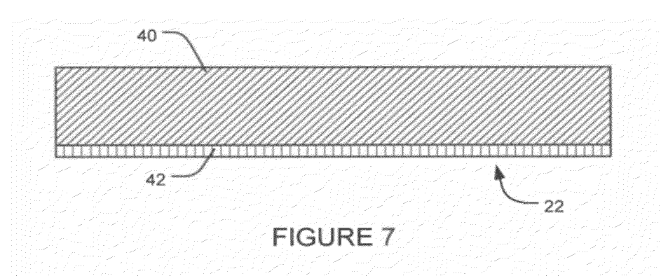

As is shown in FIGS. 1A and 1B, electrodes 22A-C extend into fluid chamber 16 and terminate in or near cell retention region 30. Each electrode 22A-C is coupled to a connector 36 for connecting electrodes 22A-C to a power supply via wires 38. Connectors 36 are made of an electrically conductive material. Apertures 37 are formed in top layer 12 to allow wires 38 to be connected to electrode connectors 36 (FIG. 3A). Wires 38 are shown in FIG. 6C.

Electrodes 22A-C may be disposed on the upper surface of bottom layer 14, or the lower surface of top layer 12, or a combination thereof. Because top layer 12 is transparent, electrodes 22A-C are visible in FIG. 1A. In some embodiments, electrode 22B is connected to two connectors 36, one on the left side of chip 10, one the right side of chip 10 (FIG. 1A). This is advantageous for automating switching voltage between electrodes 22A-22B and 22B-22C, as described further below.

Electrodes 22 are typically made of metal. In some embodiments, they may be made of an inert metal, such as platinum or gold. In cell retention region 30, electrodes 22 may have a width of 5 to 20 μm; however, this is not mandatory. According to studies conducted by the inventors, electrodes 22 having a width of 5 to 20 μm are suitable for manipulating cells or particles sized in the 5 to 20 μm range. For smaller cells (e.g. bacteria) or particles, it will be appreciated by those skilled in the art that electrodes 22 of smaller dimensions may be used. The thickness of the electrodes 22 may be 100 to 300 nm in one embodiment. In some embodiments, the thickness of the electrodes 22 is 200 nm. In one embodiment (see FIG. 7, which is not drawn to scale), the electrodes 22 comprise a first layer 40 (e.g., platinum or gold) which is 180-nm thick and a second layer 42 (e.g. tantalum) which is 20-nm thick. The second layer 42 is disposed between the first layer 40 and one of top layer 12 or bottom layer 14 of chip 10. Tantalum is selected to be used as an adhesion layer because it tends to adhere to glass better than platinum or gold. Other suitable materials may also be used.

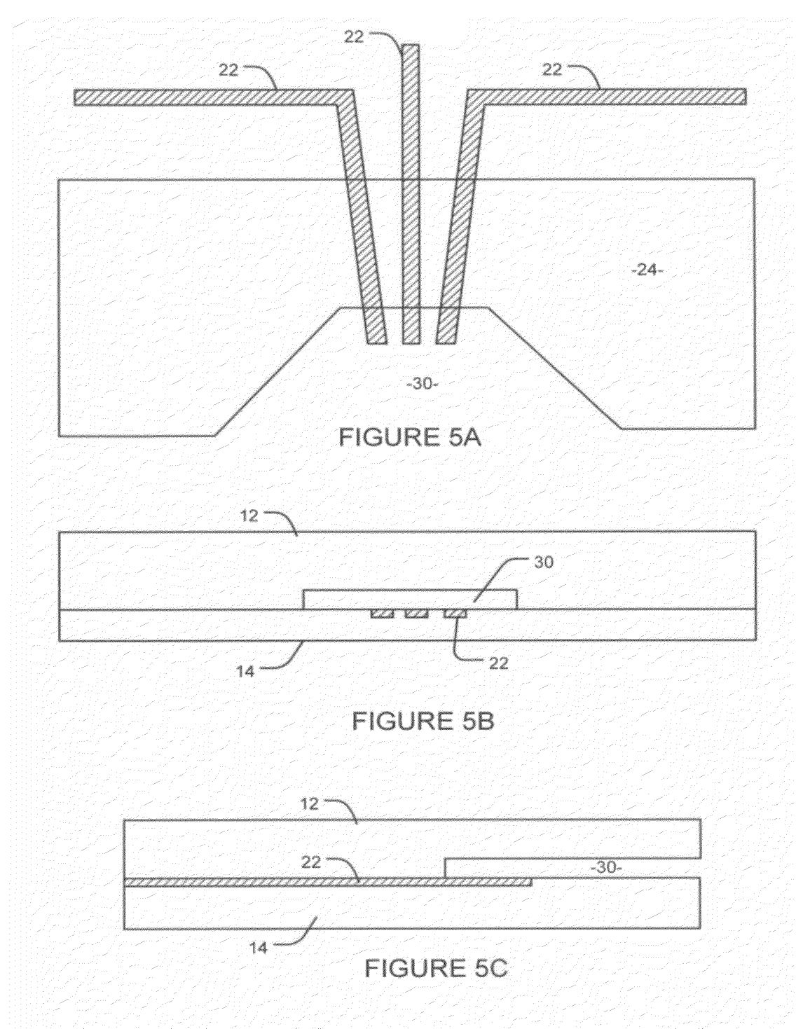

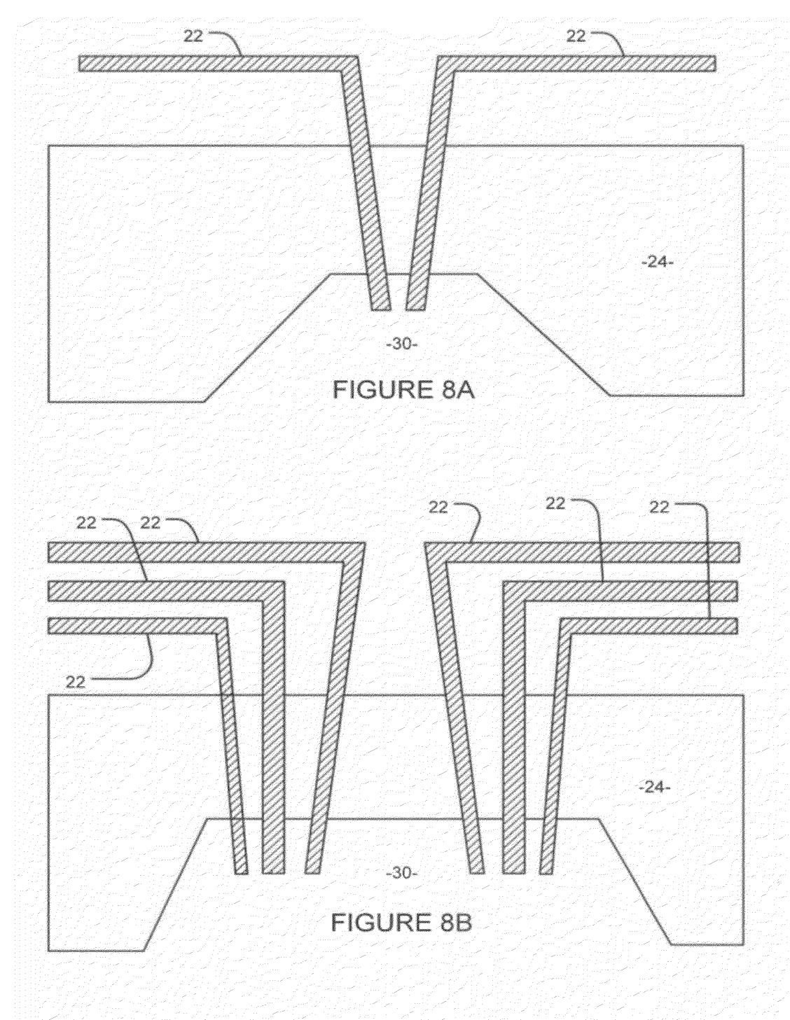

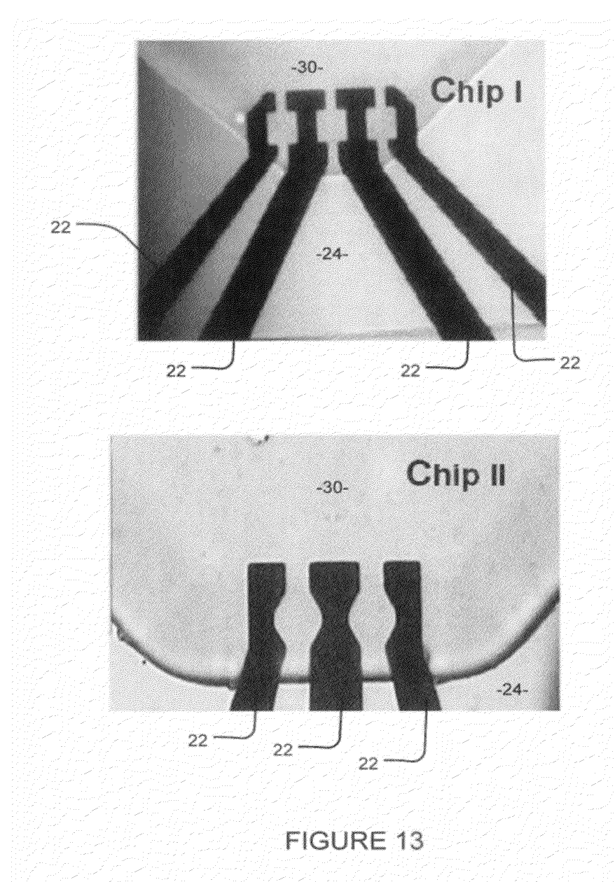

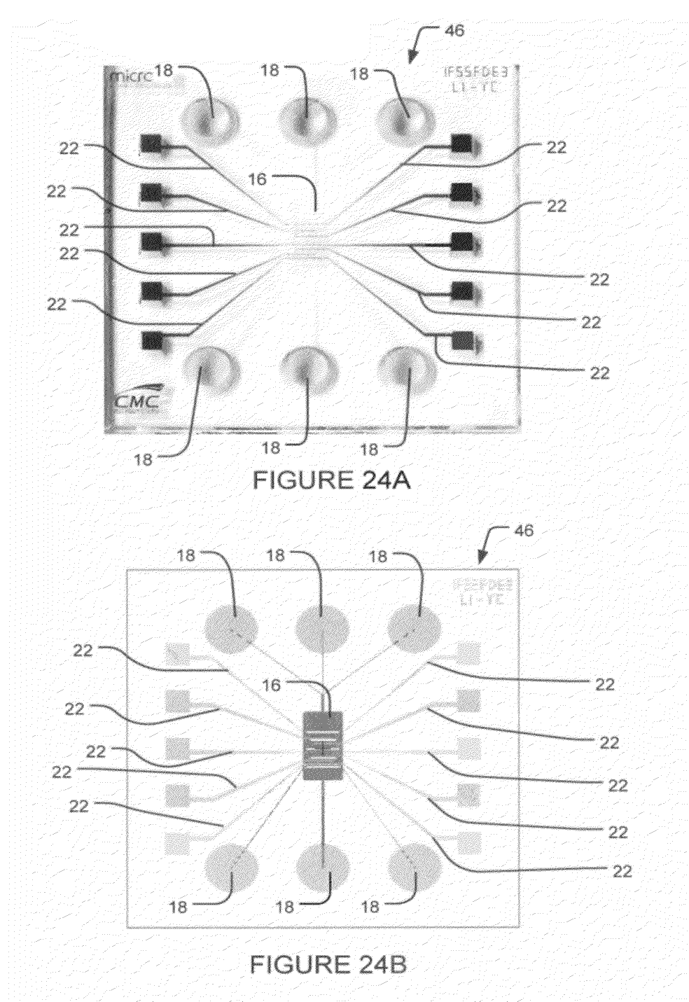

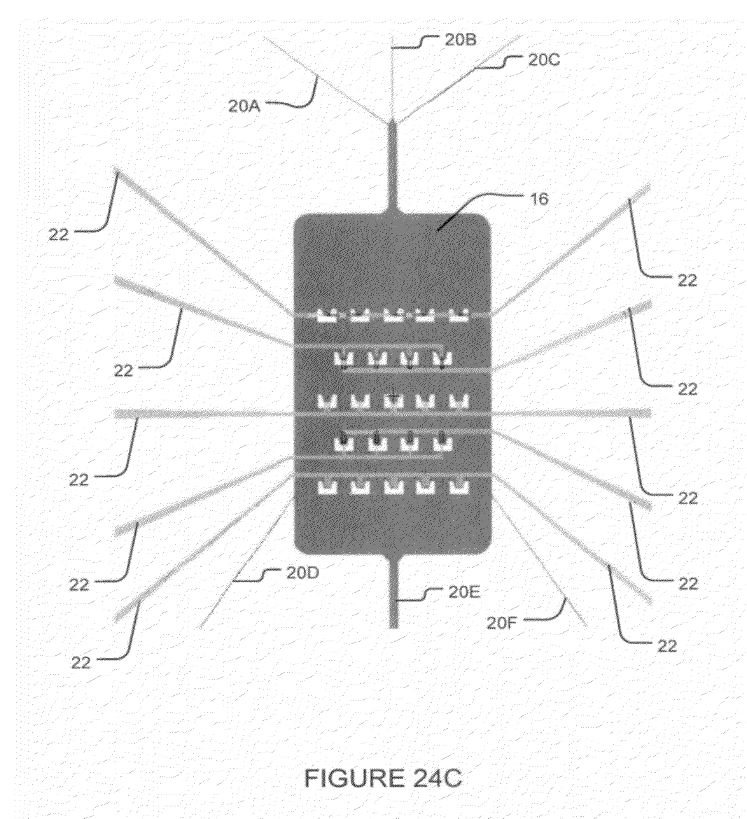

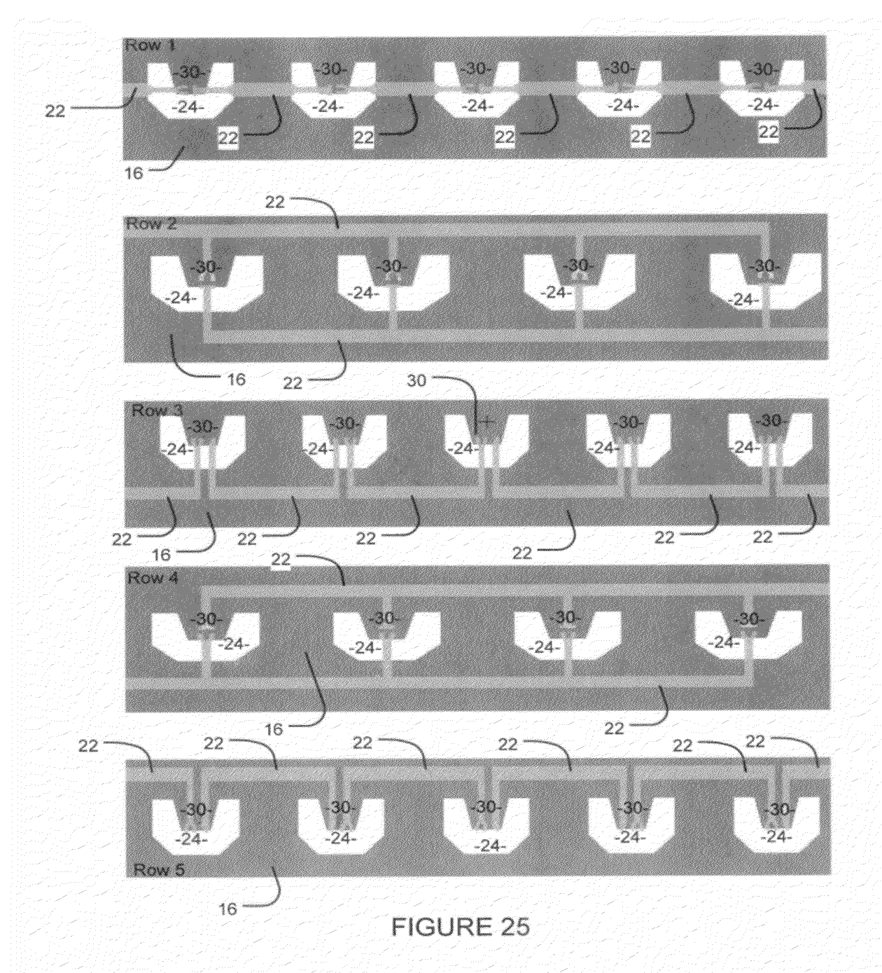

As shown in the drawings, electrodes 22 may be shaped or arranged in many different arrays. In some embodiments, there are only two electrodes 22 (FIG. 8A). In other embodiments, there are three electrodes 22 (FIG. 5A). In other embodiments, there are four or more electrodes 22 (FIG. 13, Chip I). In other embodiments, there are two sets of electrodes arrays, each set comprising two or more electrodes 22. The two sets are spaced apart in cell retention region 30 and are intended for manipulating two separate single cells in cell retention region 30 (FIG. 8B). In some embodiments, there may be an array of electrodes 22, arranged in tandem or parallel, or both (FIGS. 24 and 25).

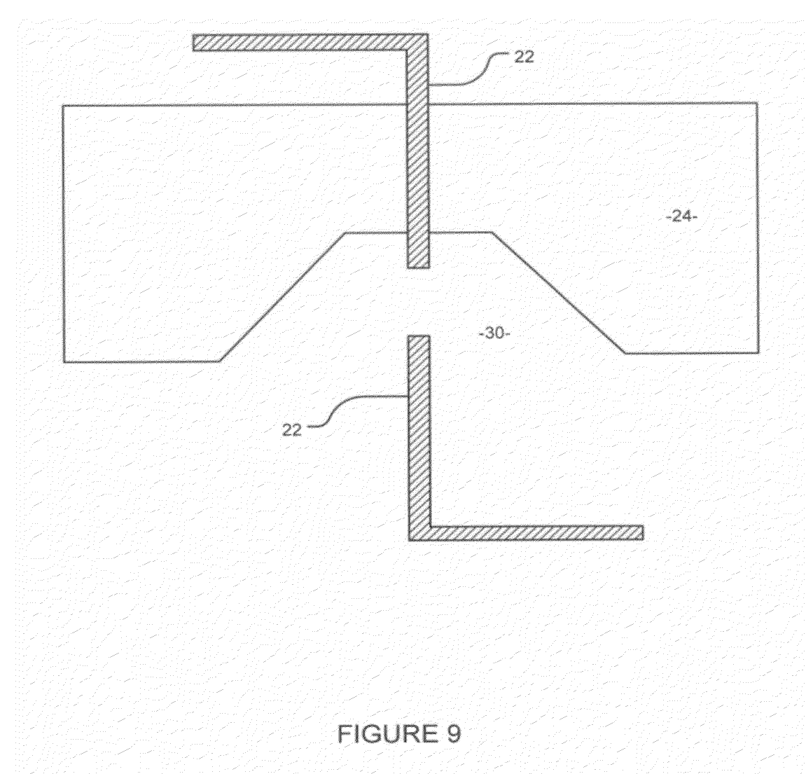

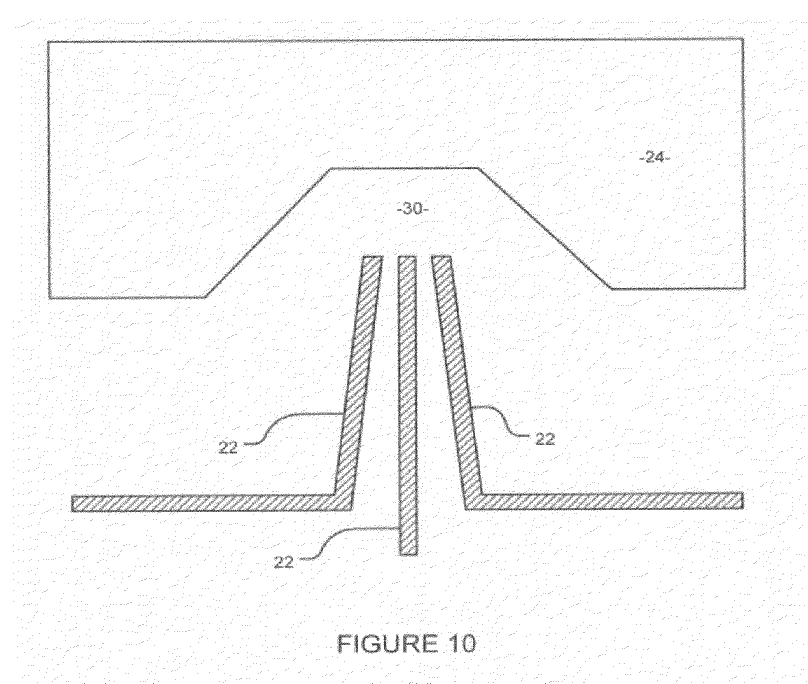

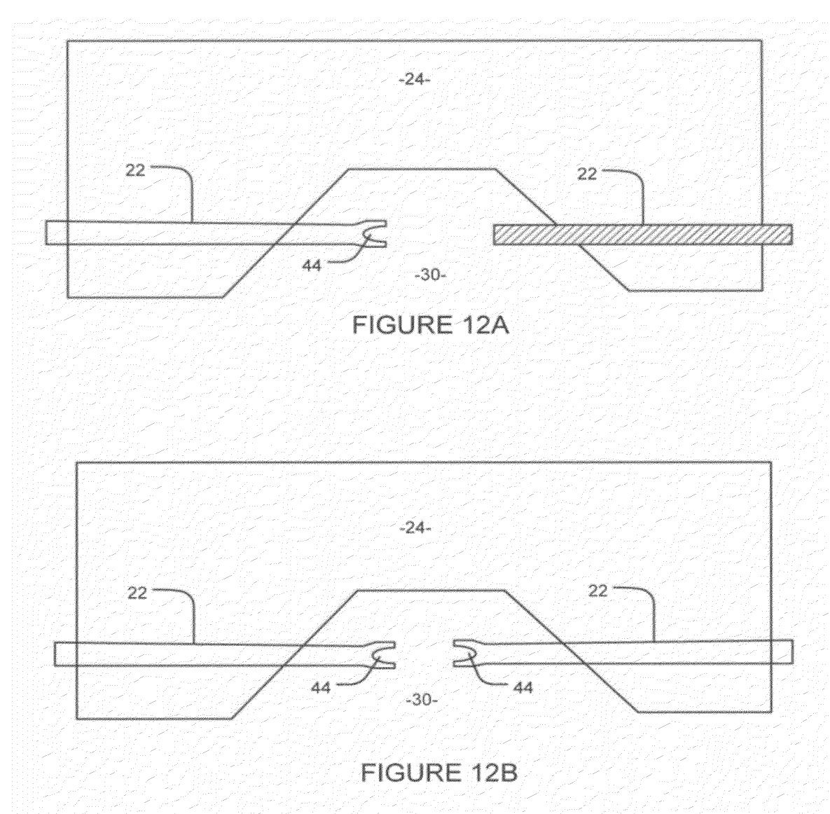

In some embodiments, electrodes 22 extend into cell retention region 30 from cell retention structure 24 (FIGS. 5A, 8A, 8B). In other embodiments, electrodes 22 extend into cell retention region 30 from the side opposite the cell retention structure 24 (FIG. 10). In still other embodiments, electrodes 22 extend into cell retention region 30 from two opposite sides, either horizontally or vertically (FIGS. 9, 12A, 12B).

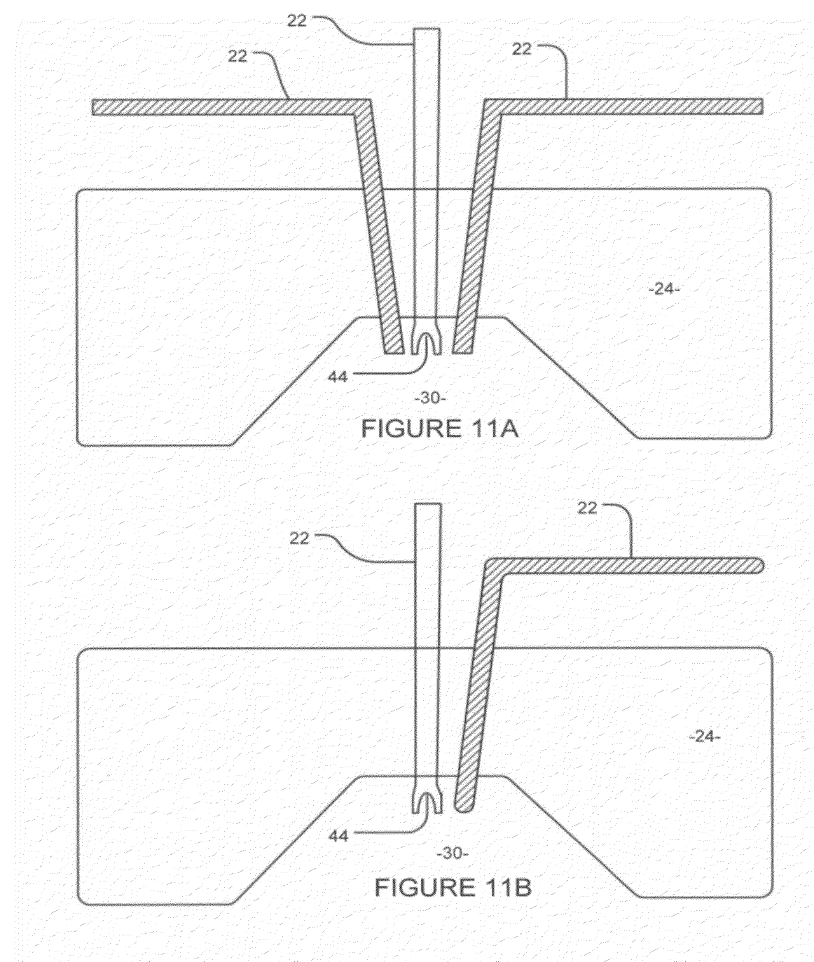

In some embodiments, the end of at least one electrode 22 extending into cell retention region 30 may be shaped like the tines of a fork (FIGS. 11A, 11B, 12A, 12B, 15). In other words, the end of electrode 22 may be shaped like the letter Y, U, or V. As described further later, the inventors have empirically determined that a single cell has the tendency to move towards or settle near a region 44 defined between the arms of the fork-shaped electrode 22 (FIG. 11A).

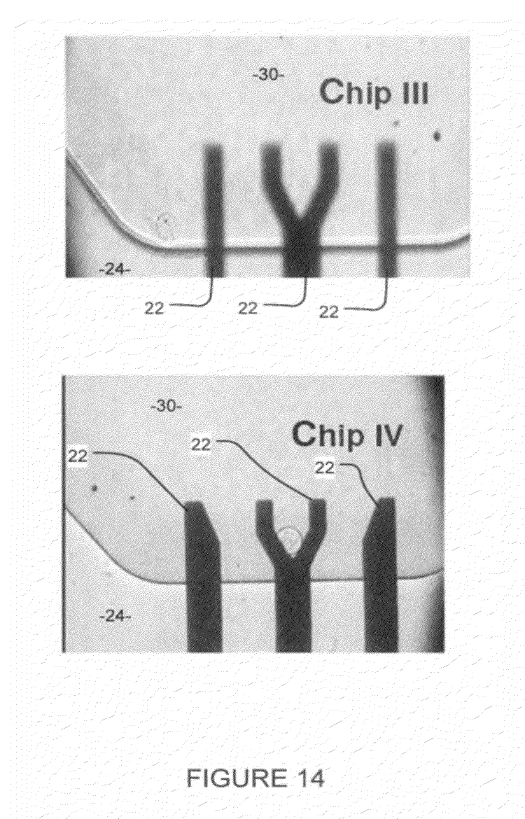

FIGS. 13 and 14 show four different DEP electrode structures of four microfluidic chips 10 (indicated as Chip I-IV). The narrowest electrode width in Chips I-IV is 10 μm (FIG. 16). In Chip I, four electrodes 22 are embedded in microfluidic chip 10. The shapes of some electrodes 22 are such that there are recessed regions and enlarged regions along the length of such electrodes 22. The tips of each electrode 22 may be generally flat or comprise other geometrical shapes, such as convex, concave, tapered, pointed or beveled. In cell retention region 30, adjacent electrodes 22 are typically spaced apart from one another by about 5 to 15 μm. For electrodes 22 of smaller dimensions, it will be appreciated by those skilled in the art that the inter-electrode distances may be adjusted accordingly. In Chip II, there are three electrodes 22. In Chip III, there are also three electrodes 22, but the middle electrode 22, which is divided into 2 branches, looks like a “fork”. The left and right electrodes 22 in Chip III have rectangular ends. In Chip IV, the middle electrode 22 has the same shape as middle electrode 22 in Chip III. However, the left and right electrodes 22 have triangularly shaped or beveled ends.

In use a chip 10 as described above can be used to controllably move cell(s) or other particle(s) within cell retention region 30 using DEP force(s) generated by electrodes 22. For example, a number of cells may be first added to one of solution reservoirs 18A, 18C. By microfluidic control, a selected cell is delivered from a reservoir 18A, 18C through a microchannel 20A, 20C to cell retention region 30 of fluid chamber 16. The cell may be selected based on the cell's physical, chemical, biological or biochemical parameters. For example, the selected cell may have a unique fluorescence property or label that distinguishes itself from the rest of the cells. Methods for selecting and moving a cell into a cell retention region 30 or similar structures using microfluidic techniques are described in: International Patent Application Publication WO 2006/007701: Paul C H Li, Fundamentals of Microfluidics and Lab on a chip for Biological Analysis and Discovery, CRC Press. 2010, 1-398; XiuJun Li, Xiaoyan Xue, Paul C. H. Li, Real-time detection of the early event of cytotoxicity of herbal ingredients on single leukemia cells studied in a microfluidic biochip, Integrative Biology, 2009, 1, 90-98: Xiujun Li, Victor Ling, Paul C. H. Li, Same-Single-Cell Approach (SASCA) for the Study of Drug Efflux Modulation of Multidrug Resistant Cells Using a Microfluidic Chip, Anal. Chem, 2008. 80, 4095-4102; James X. J. Li and Paul C. H. Li, Real-time monitoring of intracellular calcium of a single cardiomyocyte in a microfluidic chip pertaining to drug discovery, Electrophoresis, 2007. 28, 4723-4733; Xiujun (James) Li and Paul C. H. Li Microfluidic Selection and Retention of a Single Cardiac Myocyte, On-Chip Dye Loading, Cell contraction by Chemical Stimulation, and Quantitative Fluorescent. Analysis of Intracellular Calcium, Anal. Chem. 2005, 77, 4315-4322; Larry Peng and Paul C. H. Li, A three-dimensional flow control concept for single-cell experiments on a microchip (I): cell selection, cell retention, cell culture, cell balancing and cell scanning, Anal. Chem, 2004, 76, 5273-5281; Larry Peng and Paul C. H. Li, A three-dimensional flow control concept for single-cell experiments on a microchip (II): Fluorescein diacetate metabolism and calcium mobilization in a single yeast cell as stimulated by glucose and pH changes. Anal. Chem, 2004, 76, 5282-5292, all of which are hereby incorporated by reference in their entirety.

After a single cell or particle has been selected and moved into cell retention region 30, a DEP force is applied to the cell. This face is generated by applying a non-uniform electric field, or dielectric field, to the cell using electrodes 22. This field may be generated by applying an AC voltage to irregularly shaped electrodes 22. Generally, when a particle is subjected to a non-uniform electric field, a DEP force is exerted on the particle. This force does not require the particle to be charged. However, the strength of the DEP force depends on a number of factors, including the electrical properties of the medium (e.g., conductivity and permittivity), the particles electrical properties, the particles' shape and size, as well as the frequency and strength of the electric field. Consequently, electric fields of a particular frequency and/or voltage can manipulate particles with great selectivity. Factors that may influence the strength of DEP forces are described in: Pohl, H. A., 1978, Dielectrophoresis: The Behavior of Neutral Matter in Non-uniform Electric Fields. Cambridge University Press; Jeffrey D. Zahn, 2010. Methods in Bioengineering: Biomicrofabrication and Biomicrofluidics, Artech House Publishers, which are hereby incorporated by reference.

The DEP force may be generated by applying an AC voltage to at least two of the electrodes 22 that extend into cell retention region 30. In some embodiments, the AC voltage applied has a predetermined peak-to-peak voltage and frequency. The DEP force may be employed to controllably move the cell to a targeted location, for example, a detection window, to enable measurement of a physical or biological parameter of the cell. In some embodiments of the invention, the DEP force may be applied to the cell or other particle while introducing a fluid reagent into the cell retention region 30, or during reagent exchange.

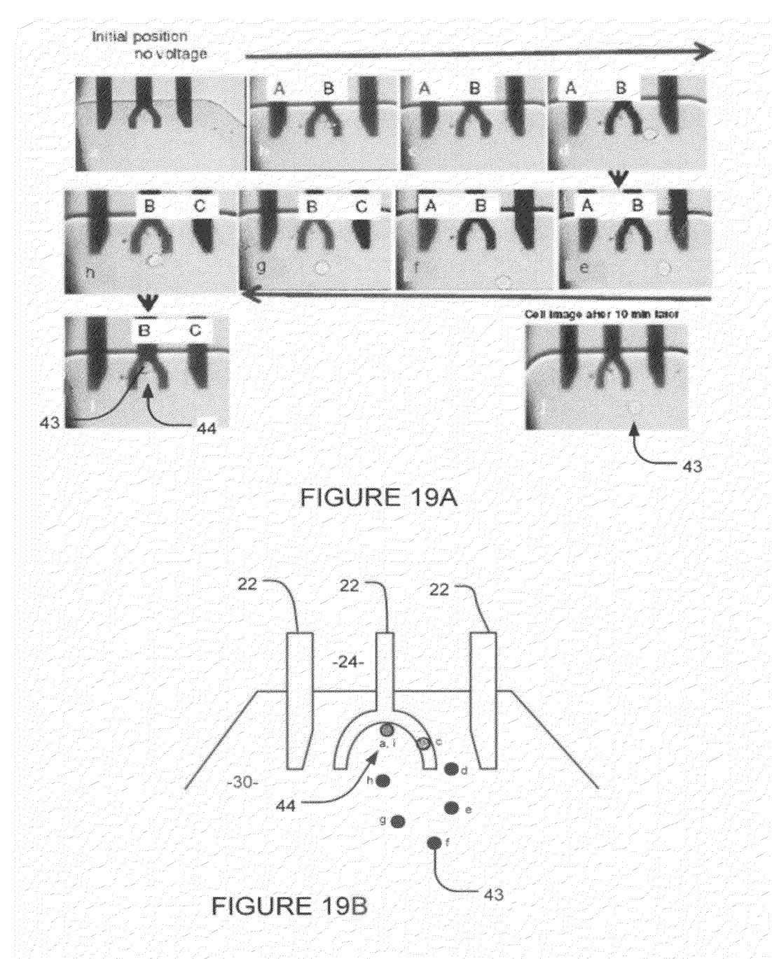

Alternating DEP forces may be applied to the cell by switching voltage between a first pair of electrodes 22 (for example, 22A and 22B) and a second pair of electrodes 22 (for example, 22B and 22C). The alternating DEP forces may be employed to controllably move the cell in first and second alternating directions. In some cases, the first direction and the second direction may be opposite directions. The alternating DEP forces may be employed, for example, to controllably move the cell from a starting location via a movement path and back to the starting location. As voltage is switched repeatedly between a first pair of electrodes 22 and a second pair of electrodes 22, the alternating DEP forces may be employed to controllably move the cell from a starting location via a movement path and back to the starting location repeatedly. The movement path may be linear, curved, or elliptical, or circular. For example. FIG. 19B, described further below, shows an elliptical movement path.

Using chip 10, it is possible to apply both a DEP force and a fluidic pressure-induced force (referred to herein as a “fluidic force”) to the cell at the same time, or at alternating times. In some embodiments, a DEP force is first applied to the cell, controllably moving the cell in a first direction; this is followed by applying a fluidic force to the cell, controllably moving the cell in a second direction. In some cases, the first direction and the second direction may be opposite directions. The alternating application of DEP and fluidic force, or the combined application of such forces, may move the cell from a starting location via a movement path and back to the starting location. The fluidic force may be applied, for example, by adding a fluid to a solution reservoir 18 in fluid communication with cell retention region 30, increased fluid head (e.g., by increasing the volume of fluid in a solution reservoir 18 or by connecting a solution reservoir 18 to a pump) increases liquid pressure and flow velocity, thus causing the cell to move at a greater velocity.

Sometimes, when the cell is subjected to both the DEP and fluidic forces and the forces are balanced, the cell may become stationary. Therefore, in some applications, DEP force and fluidic force may be used together to controllably maintain the cell at a target location in cell retention region 30.

A detection window 47 may be set at a point along the movement path of the cell such that the cell moves into and out of the detection window during each cycle of cell movement caused by DEP forces or DEP and fluidic forces. When the cell is in the detection window; a physical or biological parameter of the cell may be measured. When the cell is out of the detection window, a background parameter may be measured. As indicated above, a detection window may be defined by a beam of excitation light directed on to the surface of the chip.

The above described cell manipulation and measurement can be practiced while delivering a drug (e.g., a chemotherapeutic drug) or other reagent to cell retention region 30. The drug or other reagent may be delivered to cell retention region 30 via a solution reservoir 18 which is in fluid communication with cell retention region 30 via a microchannel 20. Typically, the drug or other reagent is delivered via middle solution reservoir 18B and microchannel 20B. Thus chip 10 can be used as a precisely controllable device for studying the affect of reagent(s) on a selected cell over time, such as the gradual accumulation of a chemotherapeutic agent by the cell.

Although chip 10 has been described above principally as a means of controllably moving a single cell or other particle by DEP forces on a microfluidic chip, it will be appreciated by a person skilled in the art that the invention could be employed to move multiple cells or other particles within cell retention region 30. For example, DEP forces may be used to cause a first cell or particle to contact a second cell or particle, such as a particle comprising a reagent, within cell retention region 30. For example, DEP forces may be used to cause a first cell to collide with a second cell to facilitate cell fusion, i.e. fusion between the first cell and the second cell. Further, as described below and illustrated in FIGS. 24A, 24B and 25, a chip 46 may be constructed comprising multiple cell retention structures 24 and cell retention regions 30 for simultaneously manipulating multiple cells within a common fluid chamber 16.

### EXAMPLES

The following examples will further illustrate embodiments of the invention in greater detail, although it will be appreciated that the invention is not limited to the specific examples.

### Example 1

This example describes a general experimental methodology for manipulating cells using a microfluidic chip 10. In one embodiment, CEM cells are sub-cultured in α-MEM medium. They are re-suspended in Hank's buffered salt solution (HESS) or α-MEM medium. It will be appreciated by those skilled in the art that other suitable solutions may also be used. Then the cells are introduced into microfluidic chip 10 by applying a drop (5 μL) of cell suspension in a cell inlet reservoir (left or right solution reservoir 18A or 18C, as indicated in FIG. 1A, but not the middle solution reservoir 18B). At least one cell is selected and delivered ftom a reservoir 18A, 18C via a microchannel 20A, 20C to cell retention region 30 within fluid chamber 16 by microfluidic flow. Then, a high-frequency, low-voltage electric field is applied between electrodes 22 (for example AB or BC, in sequence, as described further below).

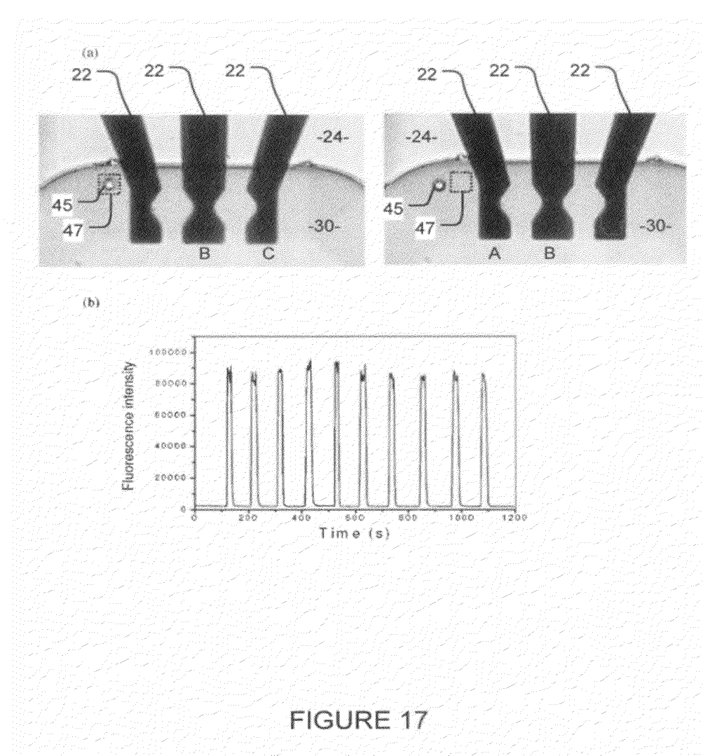

Several experimental parameters such as voltage and the frequency of the varying electric field are optimized. In some embodiments, the voltages applied are sinusoidal voltages. En some embodiments, the actual voltages are in the range of 1 to 32.5 V. The frequencies of the electric field are typically in the range of 1 to 40 MHz, although they may vary within wider limits. Then the cell is controllably moved into a detection window (for measuring the cellular fluorescence) and out of the detection window (measuring the background) by applying the voltage between electrodes AB or between BC, respectively. The detection window is set in cell retention region 30. The size of the detection window is adjustable according to the size of the cell or the particle. For example, FIG. 17 shows a smaller detection window because the bead in FIG. 17 is smaller than a typical mammalian cell.

### Example 2

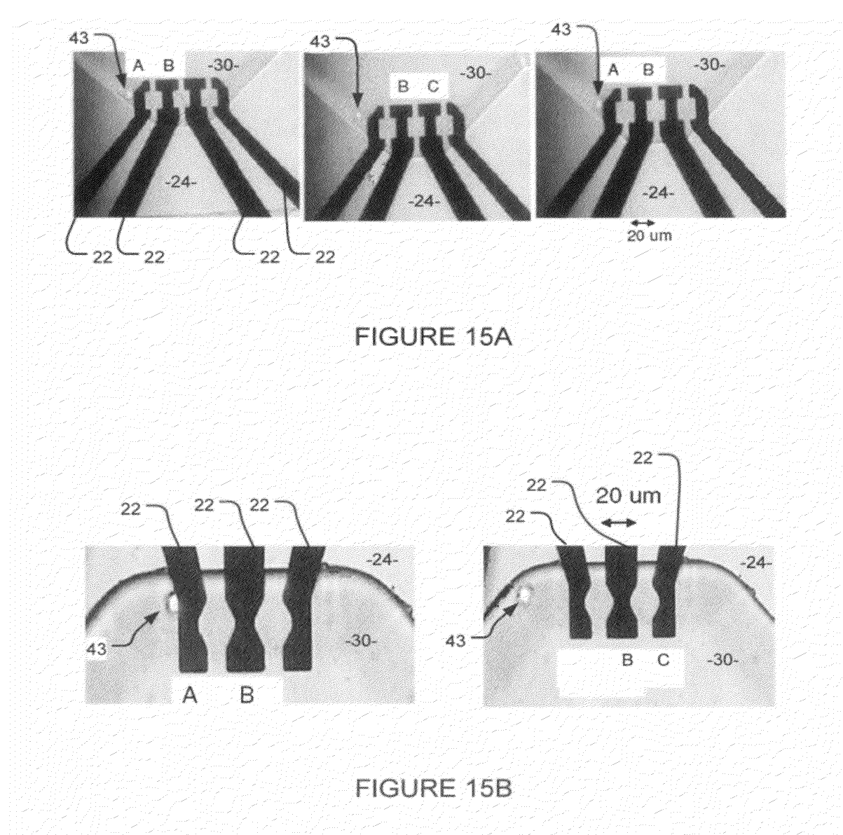

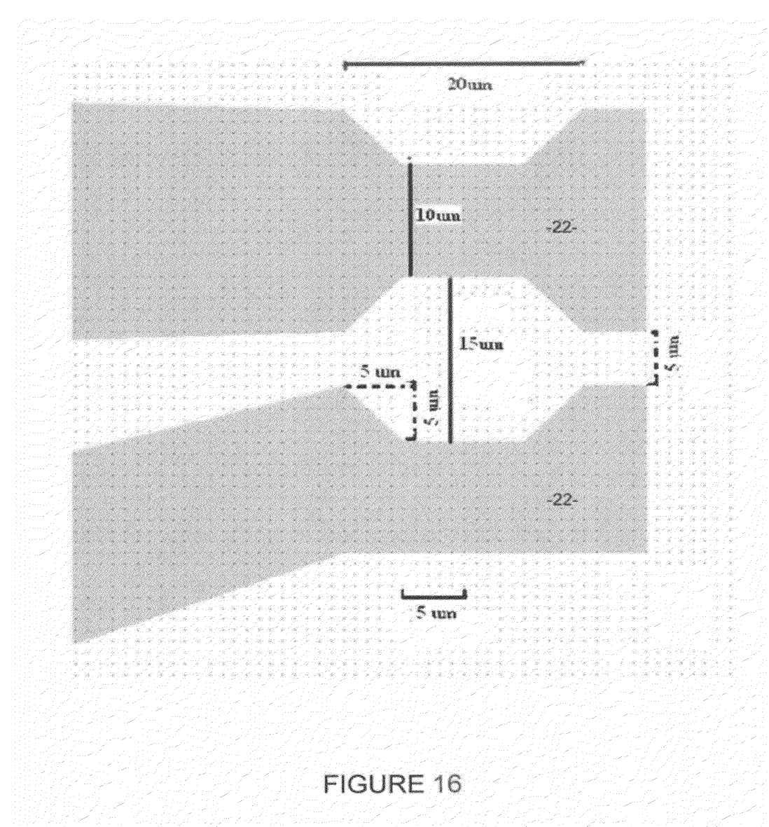

Chip I. DEP force was used to manipulate a single multidrug resistant (MDR) leukemia cell 43 (CEM VLB). The surrounding solution was HBSS; the frequency was 9 MHz and the voltage was 19 Vpp. (Vpp stands for peak-to-peak voltage.) When applying voltage between electrodes AB, cell 43 stayed close to electrode A, as shown in FIG. 15A, left panel. When the voltage was switched to electrodes BC, cell 43 was repelled away from electrode A and moved toward an edge or side wall of cell retention structure 24 (FIG. 15A, middle panel). The switching of the voltage from electrodes AB to electrodes BC was done by a programmable computer, but may also be done manually. When the voltage was switched back to electrodes AB, cell 43 was moved back close to electrode A. Therefore, the cell was caused to move in a first direction when voltage was applied to electrodes AB and in a second direction when voltage was applied to electrodes BC.

### Example 3

Chip II. DEP force was used to manipulate a single leukemia cell 43 (CEM VLB). The surrounding solution was α-MEM medium; the frequency was 9 MHz and the voltage was 14 Vpp. When applying voltage between electrodes AB, cell 43 stayed close to electrode A, as shown in FIG. 15B, left panel. When the voltage was switched to BC, cell 43 was repelled away from electrode A (FIG. 15B, right panel). And when the voltage was switched back to electrodes AB, cell 43 was moved back close to electrode A. The dimensions of the left and middle DEP electrodes in FIG. 15B is shown in a schematic diagram (FIG. 16), which is also the computer design file (L-edit) of the electrodes. The narrowest electrode width is 10 μm.

### Example 4

For proof of concept, Chip II was used to shuttle a fluorescently labeled bead 45 (in HBSS medium) and the fluorescence of bead 45 was measured. FIG. 17a shows how the bead is moved closer to electrode A and away from it. A detection window 47 is defined (see the box in dashed line, FIG. 17a). FIG. 17b shows the measurement results of bead 43 being moved back and forth for ten times, passing through detection window 47 each time. The baseline represented the background when bead 45 was moved out of detection window 47 while the peak represented the fluorescence of bead 45 when bead 45 was moved back into detection window 47. For the ten measurements, the variation is 3.5%, which indicates that bead 45 was precisely brought back to the same position at detection window 47.

### Example 5

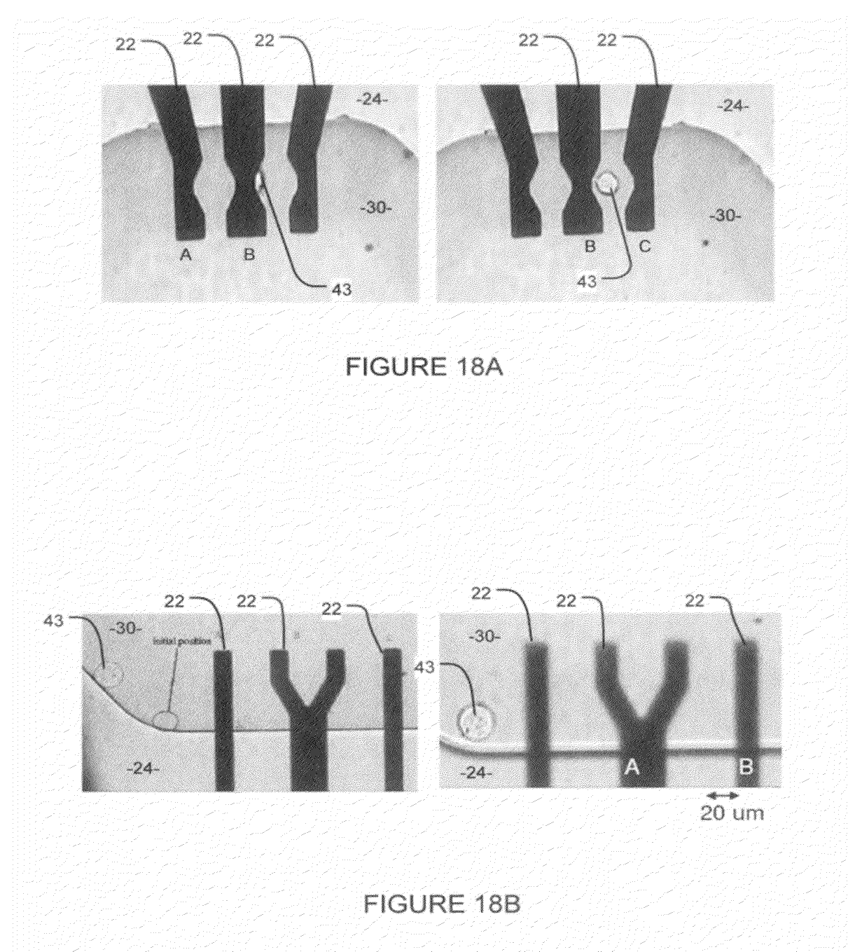

Other than α-MEM medium, HBSS buffer was also used as the cell medium. When leukemia cell 43 (CEM wild type) was manipulated at 9 MHz (14Vpp), cell 43 stayed behind the opaque electrode B when applying voltage between electrodes BC (FIG. 18A, left panel). When the voltage was switched to electrodes AB, cell 43 moved in between electrodes BC, between which the detection window could be set (FIG. 18A, right panel). Fluorescent measurement can also be performed on this cell.

### Example 6

Chip III. DEP force was used to manipulate a single leukemia cell 43 (CEM VLB). The surrounding solution was α-MEM medium. In Chip III, it was found that the previous frequency (9 MHz) used in Chip I and Chip II was not sufficient to move cell 43, let alone to reciprocatingly move it. The optimized frequency was found to be 30 MHz and the voltage was 14 Vpp. No matter where the initial position of cell 43 was, when applying voltage between electrodes AB, cell 43 inclined to stay at the left side of electrode A, as shown in FIG. 18B, right panel. When the voltage was switched to BC, cell 43 was repelled away from electrode A and moved toward the edge of cell retention region 30 (FIG. 18B, left panel). And when the voltage was switched back to AB, cell 43 was moved back close to electrode A.

### Example 7

Chip IV. DEP force was used to manipulate a single leukemia cell 43. The surrounding solution was α-MEM medium. The optimized frequency was 30 MHz and the voltage was 14 Vpp. No matter where the initial location of cell 43 was, when applying voltage between electrodes BC, cell 43 was attracted to an area 44 defined between the 2 branches of the “fork” electrode B (FIG. 19A, image i). When the voltage was switched to electrodes AB, cell 43 was repelled away from electrode B and made a movement traced in FIG. 19A (images b-f). Then when the voltage was switched back to electrodes BC, cell 43 was controllably moved back towards the center of the “fork” electrode B (FIG. 19A (images f-i) and eventually stayed in the central region 44 between the arms of the fork electrode B, i.e., at its initial position (FIG. 19A, image i). The path of movement is schematically illustrated in FIG. 19B. The path of movement is generally elliptical or circular. One cycle of movement from the starting location via the movement path and back to the starting location took 7 seconds to complete. The cell movement cycle was repeated 60 times for the same single cell 43, indicating the reproducibility of using DEP force to controllably move cell 43 back and forth or to a target location. Imaging of cell 43 after 10 min verified that cell 43 had the same appearance as initially, suggesting that cell 43 was still viable and unharmed by the DEP force.

### Example 8

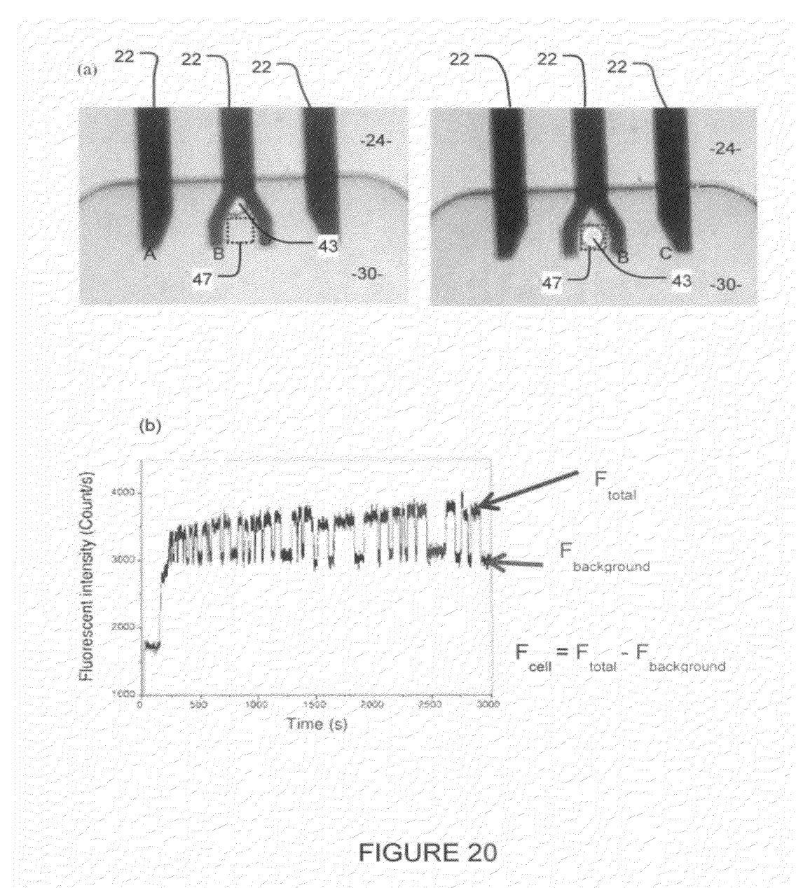

Furthermore, DEP conditions were optimized to move a single multidrug resistant cancer cell 43 during fluorescence measurement using Chip TV (FIG. 20). Cell 43 was transferred out of fluorescent detection window 47 and back into detection window 47 by switching the voltage between electrodes AB and electrodes BC. When cell 43 was moved out of detection window 47, only the background (F_{background}) was measured. When cell 43 was moved back into detection window 47, the total fluorescence (Fₜₒₜₐₗ) was measured. By background correction, the absolute fluorescence of cell 43 (F_{cell}=Fₜₒₜₐₗ−F_{background}) was obtained. At the same time, a chemo-therapeutic drug, daunorubicin (DNR) was delivered to cell retention region 30 via solution reservoir 18B and microchannel 20B. The uptake of DNR inside a single MDR cancer cell 43 (CEM VLB) was monitored. The measurement results are depicted in FIG. 20b, showing many cycles of cell fluorescence (peaks) and troughs (background). These measurement results resemble those obtained in a method previously reported and known as the same-single cell analysis method (SASCA): “Same-Single-Cell Approach (SASCA) for the Study of Drug Efflux Modulation of Multidrug Resistant Cells Using a Microfluidic Chip”, Anal. Chem. 2008, 80, 4095-4102.

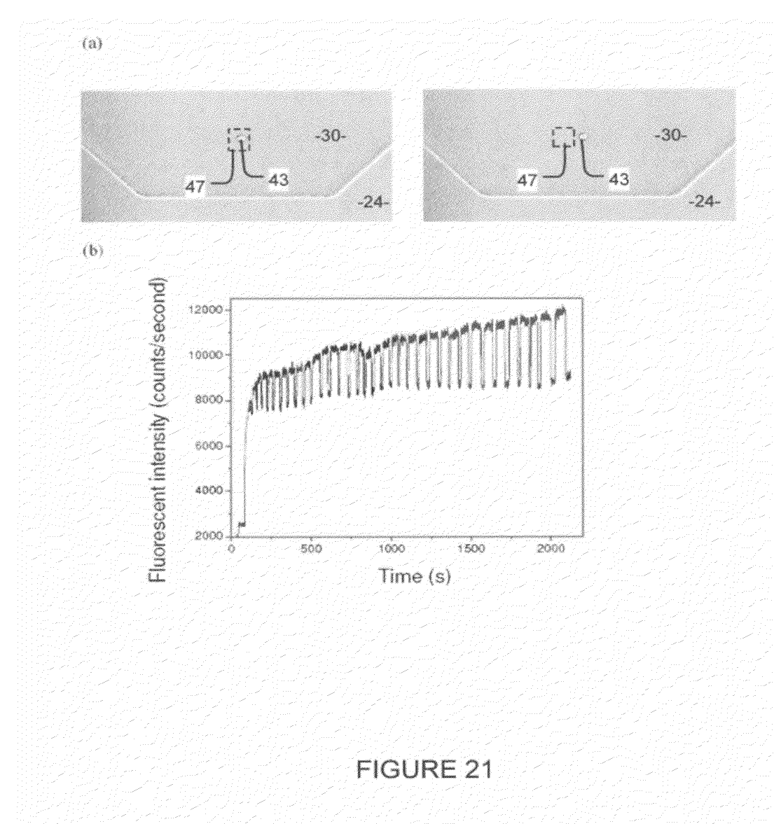

The SASCA method was performed for comparison with the DEP-based method. The SASCA method was performed in a chip without DEP electrodes. Cell 43 was moved into and out detection window 47 and at the same time DNR was delivered to cell retention region 30 (FIG. 21). In the SASCA method, cell movement was achieved by moving the chip with cell 43 which adheres to the bottom surface of cell retention region 30 in the chip. The fluorescence of cell 43 (i.e., the difference between the peaks and troughs in FIG. 21b) increased with the measurement time, indicating DNR was accumulated inside the cell.

### Example 9

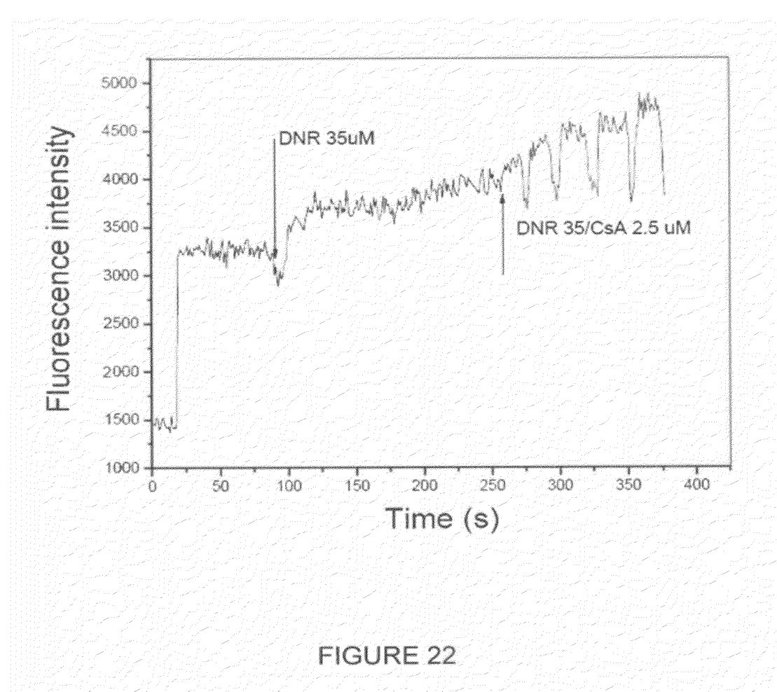

After DEP-based single cell chip 10 (Chip IV, FIG. 20) was used to move leukemia cancer cell 43 into and out of detection window 47 during fluorescence measurement for anticancer drug (for example, daunorubicin (DNR)) accumulation inside cell 43, chip 10 was further used for MDR reversal studies at an elevated temperature (35° C.). While cell 43 was being moved into and out of detection window 47 within cell retention region 30. DNR was first delivered to cell retention region 30 and then followed by cyclosporine A (CsA, a well-known MDR inhibitor). FIG. 22 shows the effect of CsA on DNR accumulation in cell 43 which was treated with DNR (35 μM). One can see that, after DNR but before CsA was delivered to cell retention region 30, there was very little DNR accumulation in cell 43, as there is no obvious differences between the peaks and the troughs during 100-200 s. However, after CsA (2.5 μM) was delivered to cell retention region 30, the intracellular DNR content started to increase, and got higher and higher during time 250-375 s.

### Example 10

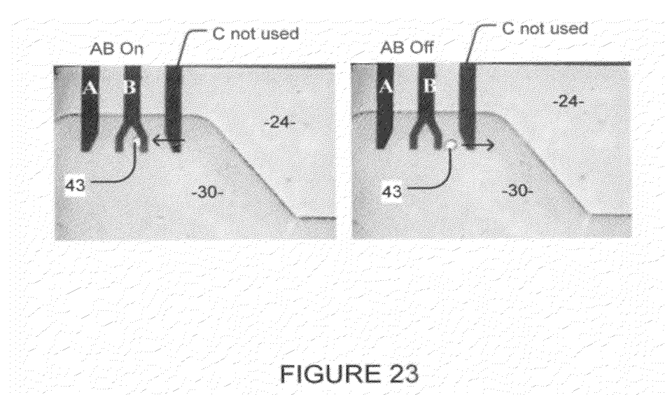

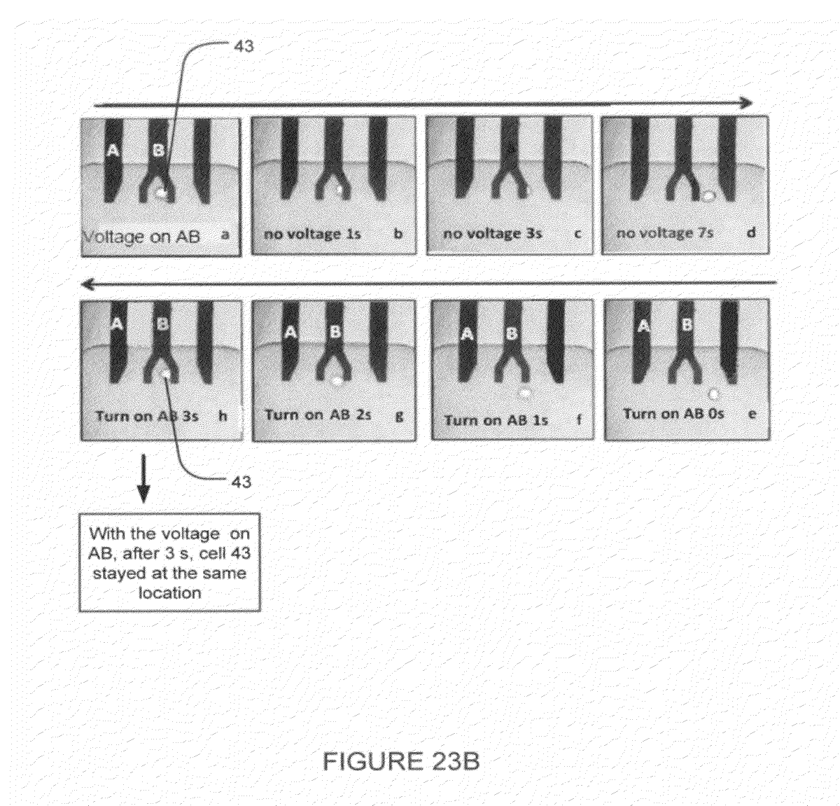

Chip IV was used to capture and move a single cell 43 by using only two electrodes 22. Instead of switching voltage between electrodes AB and BC in order to change the electric field, fluid flow itself was used to bring cell 43 away from electrodes 22 and DEP force generated by two electrodes (A and B) was used to move the cell back. FIG. 23 shows movement of cell 43 using two electrodes (A and B) as well as fluidic force. It should be noted that electrode C is not used in this experiment. When the voltage was applied to electrodes AB (FIG. 23, left panel), cell 43 was attracted to the right hand of the fork electrode B. The direction of the DEP force is indicated by the arrow in FIG. 23, left panel. When the voltage was turned off, the DEP force was gone and cell 43 was moved away by fluid flow (in a direction from left to right, as indicated by the arrow in FIG. 23, right panel). Turning back the voltage to electrodes AB, cell 43 was moved back to electrode B (in a direction from right to left). The movement of cell 43 at different time points is shown in FIG. 23B. The initial location of cell 43 is shown in FIG. 23B, image a, when the voltage to electrodes AB was turned on. When the voltage to electrodes AB was turned off, cell 43 was moved away from the fork-shaped electrode B by fluid flow (FIG. 23B, images b, c, d). When the voltage was turned on to electrodes AB, cell 43 moved back to the fork-shaped electrode B (FIG. 23B, images e, f, g, h). Cell 43 moved back to its initial location after about 3 s. After 3 s, if the voltage is continued to be applied on electrodes AB, cell 43 will remain at the same location. The path of cell movement is generally elliptical or circular. One cycle took 10 seconds to complete.

### Example 11

A high-throughput single cell chip 46 was designed and made. Chip 46 comprises multiple cell retention structures 24 within a common fluid chamber 16. Each cell retention structure 24 defines a cell retention region 30 and electrodes 22 are provided for each cell retention region 30 (FIGS. 24A, 24B, 24C and 25). With high-throughput chip 46, up to 23 single cells can be simultaneously trapped and controllably moved for fluorescence measurements at the same time. In this case, more comparable and efficient single-cell measurements may be achieved.

FIG. 24A shows a photographic image of chip 46. FIG. 24B is a schematic drawing of chip 46. FIG. 24C shows a close-up of fluid chamber 16 of chip 46. FIG. 25 is an enlarged view of the five rows of cell retention structures 24 of chip 46. Chip 46 comprises 6 solution reservoirs 18, a fluid chamber 16, and five rows of cell retention structures 24. Fluid chamber 16 is in fluid communication with solution reservoirs 18 via microchannels 20A-E (FIG. 24C). Each row of cell retention structures are provided with a different configuration of electrodes 22 for applying DEP forces to capture, manipulate or move single cells. The dimension of each cell retention structure 24 is 250*250 μm in size. As shown in FIG. 25, electrodes 22 in row 1 are aligned horizontally with cell retention structures 24. In row 2, electrodes 22 are vertically aligned. In row 3, the configuration of electrodes 22 in each cell retention structure 24 is similar to Chip IV which is described above. Row 4 is the vertically flipped design as row 2. The electrodes in row 5 and row 3 have the same shape as well except that they are oriented in opposite directions. Each of rows 1, 3, and 5 has two terminal electrodes 22, one on the left, one on the right, which are connected to connectors 36. The other electrodes 22 in between the two terminal electrodes 22 are not physically connected to the terminal electrodes 22; however, electric current may travel between these electrodes 22 via the medium in the fluid chamber which is typically conductive.

As will be apparent to those skilled in the art in the light of the foregoing disclosure, many alterations and modifications are possible in the practice of this invention without departing from the spirit or scope thereof.

### REFERENCES

1. (a) Larry Peng and Paul C. H. Li, “A three-dimensional flow control concept for single-cell experiments on a microchip (1): cell selection, cell retention, cell culture, cell balancing and cell scanning”, Anal. Chem., 2004, 76, 5273-5281. (b) Larry Peng and Paul C. H. Li, “A three-dimensional flow control concept for single-cell experiments on a microchip (II): Fluorescein diacetate metabolism and calcium mobilization in a single yeast cell as stimulated by glucose and pH changes”, Anal. Chem. 2004, 76. 5282-5292, with errata published in Anal. Chem. 2004, 76, 7400. (c) Li, X. J.; Li, P. C. H. Microfluidic selection and retention of a single cardiac myocyte, on-chip dye loading, cell contraction by chemical stimulation, and quantitative fluorescent analysis of intracellular calcium. Analytical Chemistry 2005, 77 (14), 4315-4322.
2. (a) Peng, X. Y. L.; Li, P. C. H. Extraction of pure cellular fluorescence by cell scanning in a single-cell microchip. Lab on a Chip 2005, 5 (11). 1298-1302. (b) Xiujun Li, Victor Lintz, Paul C. H. Li. “Same-Single-Cell Approach (SASCA) for the Study of Drug Efflux Modulation of Multidrug Resistant Cells Using a Microfluidic Chip”, Anal. Chem. 2008, 80, 4095-4102.
3. (a) Doh. I.; Cho, Y. A continuous cell separation chip using hydrodynamic dielectrophoresis (DEP) process. Sensors and Actuators a-Physical 2005, 121 (1), 59-65; (b) Vahey, M. D.; Voldman. J., An Equilibrium Method for Continuous-Flow Cell Sorting Using Dielectrophoresis. Anal. Chem. 2008, 80 (9), 3135-3143.
4. Auerswald, J.; Knapp, H. P. Quantitative assessment of dielectrophoresis as a micro fluidic retention and separation technique for beads and human blood erythrocytes. Microelectronic Engineering 2003, 67-8, 879-886.
5. Gonzalez, C. F.; Remcho, V. T. Harnessing dielectric forces for separations of cells, fine particles and macromolecules. Journal of Chromatography A 2005. 1079 (1-2), 59-68.
6. Muller, T.; Gradl, G.; Howitz, S.; Shirley, S.; Schnelle, T.; Fuhr, G., A 3-D microelectrode system for handling and caging single cells and particles. Biosensors & Bioelectronics 1999, 14 (3), 247-256.
7. Li, Y. L.; Dalton, C.; Crabtree, H. J.; Nilsson, G.; Kaler. K. V. I. S., Continuous dielectrophoretic cell separation microfluidic device. Lab on a Chip 2007, 7 (2), 239-248.
8. Hunt, T. P.; Westervelt, R. M. Dielectrophoresis tweezers for single cell manipulation. Biomedical Microdevices 2006, 8 (3), 227-230.
9. Baek, S. H.; Chang. W.-J.; Baek, J.-Y.; Yoon, D. S.; Bashir. R.; Lee, S. W., Dielectrophoretic Technique for Measurement of Chemical and Biological Interactions. Analytical Chemistry 2009, 81 (18), 7737-7742.
10. Borgatti, M.; Rizzo, R.; Mancini, I.; Fabbri, E.; Baricordi, O.; Gambari, R., Antibody-antigen interactions in dielectrophoresis buffers for cell manipulation on dielectrophoresis-based Lab-on-a-chip devices. Minerva Biotecnologica 2007, 19 (2), 71-74.
11. James X. J. Li and Paul C. H. Li. Real-time monitoring of intracellular calcium of a single cardiomyocyte in a microfluidic chip pertaining to drug discovery. Electrophoresis, 2007, 28, 4723-4733.
12. XiuJun Li, Xiaoyan Xue, Paul C. H. Li, Real-time detection of the early event of cytotoxicity of herbal ingredients on single leukemia cells studied in a microfluidic biochip, Integrative Biology. 2009, 1, 90-98.

What is claimed is:

1. A microfluidic device comprising:
a fluid chamber comprising a particle retention region for retaining at least one particle; and
a plurality of electrodes positioned proximate to said particle retention region for applying a dielectrophoretic (DEP) force to said particle to controllably move said particle within said particle retention region.

2. A microfluidic device according to claim 1, wherein said plurality of electrodes are capable of applying alternating DEP forces to move said particle in multiple directions.

3. A microfluidic device according to claim 1, wherein said device comprises a microfluidic flow system for controllably moving said particle within said fluid chamber by fluidic force in addition to said DEP force.

4. A microfluidic device according to claim 3, wherein said microfluidic flow system comprises at least one fluid source in fluid communication with said fluid chamber.

5. A microfluidic device according to claim 4, wherein said fluid source comprises a plurality of fluid reservoirs and a plurality of microchannels connecting said fluid reservoirs to said fluid chamber.

6. A microfluidic device according to claim 3, wherein said DEP force generated by said plurality of electrodes is adapted to move said particle in a first direction and said fluidic force generated by said flow system is adapted to move said particle in a second direction.

7. A microfluidic device according to claim 6, wherein said DEP force and said fluidic force are adapted to move said particle in alternating directions.

8. A microfluidic device according to claim 3, wherein said DEP force and said fluidic force are controllable in combination to retain said particle at a target location within said cell retention region.

9. A microfluidic device according to claim 8, wherein said target location is proximal to one of said electrodes.

10. A microfluidic device according to claim 1, wherein said plurality of electrodes comprise a first electrode and a second electrode for applying a DEP force in a first direction.

11. A microfluidic device according to claim wherein said plurality of electrodes comprise a first electrode, a second electrode and a third electrode, wherein said first and second electrodes are controllable for applying a first DEP force, and said second and third electrodes are controllable for applying a second DEP force.

12. A microfluidic device according to claim 1, wherein at least one of said electrodes is fork-shaped.

13. A microfluidic device according to claim 1, wherein at least one of said electrodes is generally Y-shaped.

14. A microfluidic device according to claim 13, wherein said at least one of said electrodes moves said particle towards an area defined between spaced segments of said Y-shaped electrode.

15. A microfluidic device according to claim 1, wherein at least one of said electrodes has a tapered end extending into said particle retention region.

16. A microfluidic device according to claim 1, wherein said electrodes are formed from an inert metal.

17. A microfluidic device according to claim 1, wherein said electrodes are formed from gold or platinum.

18. A microfluidic device according to claim 1, wherein said electrodes comprises a layer of tantalum.

19. A microfluidic device according to claim 1, wherein said at least one particle is a cell.

20. A microfluidic device according to claim 1, wherein said at least one particle is a single cell.

21. A microfluidic device according to claim 1, wherein said particle retention region is partially defined by a side wall of a cell retention structure located within said fluid chamber.

22. A microfluidic device comprising:
a fluid chamber comprising a plurality of particle retention structures, each particle retention structure defining a particle retention region for retaining at least one particle therein; and
a plurality of electrodes extending into each of said particle retention regions for applying a DEP force to controllably move said at least one particle therein.

23. A microfluidic device according to claim 22, wherein said plurality of particle retention structures are arranged in a row.

24. A microfluidic device according to claim 22, wherein said plurality of particle retention structures are arranged in an array.

25. A method of controllably moving at least one particle in a microfluidic device comprising:
a) positioning said particle in a fluid particle retention region of said device; and
b) applying a DEP force to said particle to controllably move said particle within said particle retention region.

26. A method according to claim 25, further comprising applying a fluidic force to said particle within said particle retention region.

27. A method according to claim 25, wherein said at least one particle comprises a single cell suspended in said fluid.

28. A method according to claim 25, wherein said applying said DEP force comprises applying alternating DEP forces.

29. A method according to claim 25, comprising moving said particle in alternating directions.

30. A method according to claim 25, comprising moving said particle from a starting location to an ending location via a movement path.

31. A method according to claim 30, wherein said movement path passes through a detection window at least once.

32. A method according to claim 31, wherein said particle is a cell and said method comprises measuring a physical or biological parameter of said cell when said cell is in said detection window.

33. A method according to claim 32, comprising measuring a background parameter of said fluid when said cell is not present in said detection window.

34. A method according to claim 26, wherein said particle is a single cell and said method comprises moving said cell from a starting location to an ending location via a movement path, wherein said fluidic force is applied to said cell during said moving.

35. A method according to claim 34, wherein said applying a fluidic force comprises adding a fluid to a solution reservoir in fluid communication with said particle retention region.

36. A method according to claim 25, wherein said positioning comprises moving said particle from a solution reservoir in fluid communication with said particle retention region.

37. A method according to claim 34, further comprising delivering a reagent to said cell by adding said reagent to a solution reservoir in fluid communication with said particle retention region.

38. A method according to claim 30, wherein said movement path is linear.

39. A method according to claim 30, wherein said movement path is curved.

40. A method according to claim 30, wherein said movement path reciprocates between said starting and ending locations, and wherein said movement path passes through a detection window positioned on said microfluidic device.
